(19) United States
(12) Patent Application Publication
(10) Pub. No.: US 2021/0236023 A1
(43) Pub. Date: Aug. 5, 2021

(54) TECHNOLOGY ADAPTED TO ENABLE IMPROVED COLLECTION OF INVOLUNTARY EYELlD MOVEMENT PARAMETERS, INCLUDING COLLECTION OF EYELlD MOVEMENT PARAMETERS TO SUPPORT ANALYSIS OF NEUROLOGICAL FACTORS

(71) Applicant: SDIP HOLDINGS PTY LTD, Victoria (AU)

(72) Inventors: Murray Johns, Queenscliff, NSW (AU); Scott Coles, Queenscliff Queenscliff, New S (AU); Trefor Morgan, Queenscliff, NSW (AU)

(21) Appl. No.: 16/972,535

(22) PCT Filed: Jun. 5, 2019
(86) PCT No.: PCT/AU2019/050576

§ 371 Date: Dec. 4, 2020

(30) Foreign Application Priority Data
Jun. 5, 2018 (AU) 2018902016
Oct. 23, 2018 (AU) 2018904026
Oct. 23, 2018 (AU) 2018904027
Oct. 23, 2018 (AU) 2018904028
Oct. 27, 2018 (AU) 2018904076
Nov. 13, 2018 (AU) 2018904312
Jan. 25, 2019 (AU) 2019900229

(51) Int. Cl. A61B 5/11; A61B 5/00; A61B 5/1171

(52) U.S. Cl. CPC A61B 5/1103; A61B 5/0077; A61B 5/1128; A61B 5/1176; A61B 2560/0247; A61B 5/6898; A61B 5/742; A61B 5/4082; A61B 5/7221

(57) ABSTRACT

Technology is adapted to enable improved collection of involuntary eyelid movement parameters, including collection of eyelid movement parameters to support analysis of neurological factors. For example, this may include methods and systems configured to enable improved analysis of involuntary eyelid movement parameters, including diagnosis of subject neurological conditions and/or other subject attributes from analysis of involuntary eyelid movement parameters. Some embodiments relate to testing, which provide a standardized environment for collection of involuntary eyelid movement data thereby to reduce influence of variable factors, which affect involuntary eyelid movement. For example, the standardized environment influences the subject to adopt a controlled cognitive and/or physiological state, thereby to improve comparability of test results. In some cases, the controlled test parameters include a test parameter, which influences the subject to voluntarily maintain a substantially consistent gaze detection, thereby to minimize eye movement.

### CROSS-REFERENCE TO RELATED APPLICATIONS

This application is a national phase entry under 35 U.S.C. § 371 of International Patent Application PCT/AU2019/050576, filed Jun. 5, 2019, designating the United States of America and published as International Patent Publication WO 2019/232579 A1 on Dec. 12, 2019, which claims the benefit under Article 8 of the Patent Cooperation Treaty to Australian Patent Application Serial No. 2018902016, filed Jun. 5, 2018, Australian Patent Application Serial No. 2018904026, filed Oct. 23, 2018, Australian Patent Application Serial No. 2018904027, filed Oct. 23, 2018, Australian Patent Application Serial No. 2018904028, filed Oct. 23, 2018, Australian Patent Application Serial No. 2018904076, filed Oct. 27, 2018, Australian Patent Application Serial No. 2018904312, filed Nov. 13, 2018, and Australian Patent Application Serial No. 2019900229, filed Jan. 25, 2019.

### TECHNICAL FIELD

The present disclosure relates, in various embodiments, to technology adapted to enable improved collection of involuntary eyelid movement parameters, including collection of eyelid movement parameters to support analysis of neurological factors. Embodiments improve collection techniques by providing standardized testing methodologies, which facilitate identification of neurological conditions substantially independent of unwanted blink influencing factors. For example, this may include methods and systems configured to enable improved analysis of involuntary eyelid movement parameters, including diagnosis of subject neurological conditions and/or other subject attributes from analysis of involuntary eyelid movement parameters. Some embodiments relate to testing approaches, frameworks and hardware systems that are configured to enable improved collection and analysis of eyelid movement parameter data, enabling enhanced research, diagnosis and other practices. While some embodiments will be described herein with particular reference to those applications, it will be appreciated that the present disclosure is not limited to such a field of use, and is applicable in broader contexts.

### BACKGROUND

Any discussion of the background art throughout the specification should in no way be considered as an admission that such art is widely known or forms part of common general knowledge in the field.

It is known to diagnose neurological conditions from analysis of eye and/or eyelid movements. For example, U.S. Pat. No. 7,791,491 teaches a method and apparatus for measuring drowsiness based on the amplitude to velocity ratio for eyelids closing and opening during blinking as well as measuring duration of opening and closing. This enables an objective measurement of drowsiness.

Through research into relationships between eye and eyelid movement parameters and neurological conditions, opportunities for diagnosis of additional neurological conditions via analysis of eyelid movement parameters have been identified. However, seeking to realize such opportunities has revealed a need for improved techniques for collection and analysis of the parameter data.

### BRIEF SUMMARY

It is an object of the present disclosure to overcome or ameliorate at least one of the disadvantages of the prior art, or to provide a useful alternative.

One embodiment provides a method for collection of involuntary eyelid movement data from a human subject under controlled conditions, wherein the method is performed based on execution of software instructions via a hardware device having: (i) a display screen; (ii) a camera module facing in a common direction to the display screen; and (iii) an input device; the method including:
delivering, via the display screen, a subject state standardization test having controlled test parameters, wherein the subject state standardization test provides a standardized environment for collection of involuntary eyelid movement data thereby to reduce influence of variable factors, which affect involuntary eyelid movement, wherein the subject state standardization test is configured to deliver controlled stimuli to the subject for a defined test period;
collecting, via the input device, voluntary response data for the subject state standardization test, wherein the voluntary response data excludes eyelid movement;
collecting, from the subject via the camera module, data measurements representative of involuntary eyelid movement parameters during the defined test period;
processing the voluntary response data thereby to determine whether the subject's performance of the subject state standardization meets threshold performance requirements;
in the case that the voluntary response data meets threshold performance requirements, making a determination that the data measurements representative of involuntary eyelid movement parameters during the defined test period meet a collection standards requirement.

One embodiment provides a method wherein the standardized environment influences the subject to adopt a controlled cognitive and/or physiological state, thereby to enable comparison between data measurements representative of involuntary eyelid movement parameters between a first subject and a second subject substantially independent of variability responsive to the subjects' respective cognitive and/or physiological states.

One embodiment provides a method wherein the controlled test parameters include a test parameter, which influences the subject to voluntarily maintain a substantially consistent gaze detection, thereby to minimize eye movement.

One embodiment provides a method wherein the data measurements representative of involuntary eyelid movement parameters include a measure of eyelid position, thereby to enable determination parameters that define blink attributes including eyelid closure times

One embodiment provides a method wherein a facial recognition algorithm is used to enable identification of: (i) a central position on an upper eyelid on a detected face; and (ii) at least two fixed points on the detected face; wherein the two fixed points on the detected face are used to enable scaling of measurements of movement of the central position of the upper eyelid thereby to account to changes in relative distance between the user and the camera.

One embodiment provides a method including processing the data measurements representative of eyelid movement parameters thereby to determine a rate of change for one or more eyelid movement parameters during at least a subset of the time for which the subject state standardization test is delivered.

One embodiment provides a method preceding claim wherein the subset of the time for which the subject state standardization test is delivered excludes an initial time segment of the time for which the subject state standardization test is delivered.

One embodiment provides a method wherein the hardware device includes or is coupled to one or more sensors that are configured to measure environmental conditions, and the method includes:
processing input data from one of the sensors thereby to determine whether the environmental conditions meet environmental standardization requirements; and
in the case that the environmental conditions meets environmental standardization requirements, making a determination that the data measurements representative of involuntary eyelid movement parameters during the defined test period meet a further collection standards requirement.

One embodiment provides a method wherein the environmental conditions include one or more of: ambient light; ambient noise; and ambient motion.

One embodiment provides a method wherein the ambient motion includes motion of the subject, and motion in an area surrounding the subject.

One embodiment provides a method wherein the hardware device includes or is coupled to one or more sensors that are configured to measure human physiological conditions, and the method includes:
processing input data from one of the sensors thereby to determine whether the human physiological conditions meet physiological standardization requirements; and
in the case that the physiological conditions meet physiological standardization requirements, making a determination that the data measurements representative of involuntary eyelid movement parameters during the defined test period meet a further collection standards requirement.

One embodiment provides a method wherein the physiological conditions include one or more of: human movement during the subject state standardization test; human movement prior to the subject state standardization test; and subject heart rate.

One embodiment provides a method wherein the hardware device is a smartphone or tablet device.

One embodiment provides a method wherein the subject state standardization test includes a test wherein a visual artefact is displayed at a controlled location on the display screen, and the subject prompted to provide an input in response to changes in characteristics of the visual artefact.

One embodiment provides a method for analysis of eyelid parameter data from a human subject, the method including:
accessing a set of test voluntary subject response data, wherein the set of test voluntary subject response data defines a record of voluntary subject responses to stimuli delivered via a subject state standardization test having controlled test parameters, wherein the subject state standardization test is configured to deliver controlled stimuli to the subject for a defined test period;
identifying a set of test involuntary movement data associated with the set of test voluntary subject response data, wherein the set of test involuntary movement data is defined via operation of eyelid monitoring hardware during the defined test period thereby to collect, from the subject, data measurements representative of involuntary eyelid movement parameters during the defined test period;
analyzing the set of test voluntary subject response data, thereby to identify whether the voluntary subject responses fall within a predefined subject state standardization confirmation profile;
in the case that the voluntary subject responses fall within a predefined subject state standardization confirmation profile, determining that the associated set of test involuntary movement data meets a diagnostic validity requirement; and
in the case that the voluntary subject responses fall outside the predefined subject state standardization confirmation profile, determining that the associated set of test involuntary movement data fails to meet the diagnostic validity requirement.

One embodiment provides a method wherein the subject state standardization test is delivered via a hardware system having a display screen configured to deliver the stimuli as visual stimuli, one or more input devices configured to receive the voluntary subject responses to the visual stimuli.

One embodiment provides a method wherein the subject state standardization test includes a reaction time test.

One embodiment provides a method the subject state standardization test includes a test wherein a visual artefact is displayed at a controlled location on the display screen, and wherein the stimuli is defined by modification of the visual artefact.

One embodiment provides a method wherein modification of the visual artefact occurs for a predefined duration that is consistent across the stimuli.

One embodiment provides a method wherein triggering of the modification of the visual artefact occurs on an irregular basis.

One embodiment provides a method wherein the irregular basis is defined in a pseudorandom manner.

One embodiment provides a method wherein the controlled location is a static location on the display screen.

One embodiment provides a method wherein delivering the subject state standardization test includes delivering to the subject a set of instructions defining test conditions.

One embodiment provides a method wherein the subject state standardization test is delivered by a hardware system that includes one or more sensors that are configured to measure test conditions parameters thereby to enable determination of compliance with predefined test conditions requirements.

One embodiment provides a method wherein the one or more sensors include an inertial measurement unit configured to determine hardware motion parameters.

One embodiment provides a method wherein the one or more sensors include a light measurement sensor configured to determine ambient light parameters.

One embodiment provides a method wherein analyzing the set of test voluntary subject response data includes analyzing subject responses to the stimuli.

One embodiment provides a method wherein analyzing the set of test voluntary subject response data includes identifying errors of omission, wherein an error of omission is defined in the case that the subject fails to respond to a given one of the stimuli within a predefined response threshold period.

One embodiment provides a method wherein analyzing the set of test voluntary subject response data includes identifying errors of co-mission, wherein an error of co-mission is defined in the case that the subject provides a response input that does not correspond to a unique one of the stimuli.

One embodiment provides a method wherein analyzing the set of test voluntary subject response data includes analyzing reaction times.

One embodiment provides a method wherein analyzing the set of test voluntary subject response data includes as a primary measure identifying errors of omission and/or co-mission, wherein analysis of analysis of response attributes including response time is performed as an optional secondary metric.

One embodiment provides a method wherein analyzing the set of test voluntary subject response data, thereby to identify whether the voluntary subject responses fall within a predefined subject state standardization confirmation profile, is configured to predict whether the subject has for the duration of the test period adopted a standardized state of activity.

One embodiment provides a method wherein a prediction that the subject has for the duration of the test period adopted a standardized state of activity is made in the case that the subject satisfies a threshold for successful completion of the test based on a number of errors of omission and/or co-mission.

One embodiment provides a method wherein set of test involuntary movement data includes eyelid movement data.

One embodiment provides a method wherein the eyelid movement data includes a measure of blink attributes for a plurality of blink events during the test period.

One embodiment provides a method wherein the measure of blink attributes for a given blink event includes any one or more of: a time period from blink initiation to blink completion; a time period for eye closure motion; a time period during which the eye is closed; and a time period for eye re-opening motion.

One embodiment provides a method wherein the measure of blink attributes for a given blink event includes a time period for eye closure motion; a time period during which the eye is closed; and a time period for eye re-opening motion.

One embodiment provides a method wherein the measure of blink attributes for a given blink event includes measurement of a time period data and measurement of eyelid movement velocity data.

One embodiment provides a method wherein the measure of blink attributes for a given blink event includes a time period for eye closure motion; a time period during which the eye is closed; a time period for eye re-opening motion; a velocity of eye closure motion; and a velocity of eye re-opening motion.

One embodiment provides a method wherein the measure of blink attributes for a given blink event includes data enabling calculation of amplitude-to-velocity ratios.

One embodiment provides a method wherein set of test involuntary movement data includes involuntary eye movement data.

One embodiment provides a method wherein the involuntary eye movement data includes saccades.

One embodiment provides a method wherein the subject state standardization test includes delivery of stimuli configured to cause defined voluntary eye movement data thereby to enable identification of involuntary eye movement data.

One embodiment provides a method wherein set of test involuntary movement data includes eyelid movement data and eye movement data.

One embodiment provides a method wherein the eyelid monitoring hardware includes an image capture unit.

One embodiment provides a method wherein the image capture unit is provided on via hardware device that includes a display screen, wherein the subject state standardization test is rendered on that display screen.

One embodiment provides a method wherein the image capture unit is coupled to a processing unit, wherein the processing unit is configured to execute software instructions that cause processing of image data obtained via the image capture unit, wherein the processing of image data includes image-based identification of one or more eyes of the subject, and for at least one eye, image-based identification of eyelid movement.

One embodiment provides a method wherein the eyelid monitoring hardware includes a wearable eyelid parameter monitoring device.

One embodiment provides a method wherein the wearable eyelid parameter monitoring device includes spectacles having sensors configured to measure eyelid movement data.

One embodiment provides a method wherein the sensors include hardware configured to perform infrared reflectance oculography.

One embodiment provides a method including performing analysis of the test involuntary movement data.

One embodiment provides a method wherein performing analysis of the test involuntary movement data occurs only where that data is determined to meet the diagnostic validity requirement.

One embodiment provides a method wherein the analysis includes categorisation of blink events into a set of predefined classes.

One embodiment provides a method wherein the classes are defined by blink parameter definitions.

One embodiment provides a method wherein the blink parameter definitions include a blink period, such that the blink events are categorised into a predefined set of classes with each class being defined by a respective blink period time range.

One embodiment provides a method wherein the analysis includes performing a statistical analysis of blink characteristics as a function of time.

One embodiment provides a method wherein the analysis includes determining rate of change attributes of blink event parameters.

One embodiment provides a method wherein the rate of change attributes of blink event parameters includes any one or more of: a point in time during the test period when a change commenced; a rate of change; and an acceleration or deceleration of rate of change over time.

One embodiment provides a method wherein the blink event parameters include: blink time period parameters; blink motion rate parameters; and blink frequency parameters.

One embodiment provides a method wherein the analysis includes categorisation of blink events into a set of predefined classes, and wherein performing the statistical analysis of blink characteristics as a function of time includes determining changes in frequency of blinks categorized into each of the set of predefined classes as a function of time.

One embodiment provides a method wherein, in the case that the voluntary subject responses fall within the predefined subject state standardization confirmation profile, determining that the associated set of test involuntary movement data meets a diagnostic validity requirement, and analyzing the set of test involuntary movement data thereby to diagnose a condition in the subject.

One embodiment provides a method including diagnosing a condition in the subject based on the analysis of the test involuntary movement data in combination with other data.

One embodiment provides a method wherein the other data includes data collected in response to the subject state standardization test.

One embodiment provides a method wherein the step of diagnosing includes comparing results for the subject data representative of one or more sets of benchmark results.

One embodiment provides a method wherein the step of diagnosing includes identifying presence of an eyelid based biomarker.

One embodiment provides a method wherein, in the case that the voluntary subject responses fall within the predefined subject state standardization confirmation profile, determining that the associated set of test involuntary movement data meets a diagnostic validity requirement, and adding the test involuntary movement data to a larger data set that is maintained for identification of condition-representative biomarkers in eyelid movement data.

One embodiment provides a method wherein, in the case that the voluntary subject responses fall within a predefined subject state standardization confirmation profile, determining that the associated set of test involuntary movement data meets a diagnostic validity requirement, and adding the test involuntary movement data to a larger data set, and performing analysis of the larger data set thereby to perform candidate identification for one or more condition-representative biomarkers in eyelid movement data.

One embodiment provides a method wherein the larger data set includes further sets of test involuntary movement data determined to meet the diagnostic validity requirement by the method as discussed herein.

One embodiment provides a method wherein, in the case that the voluntary subject responses fall within the predefined subject state standardization confirmation profile, analyzing the associated set of test involuntary movement data thereby to, based on changes in blink parameters, perform a determination of subject maintenance of alertness.

One embodiment provides a method wherein, in the case that the voluntary subject responses fall within the predefined subject state standardization confirmation profile, analyzing the associated set of test involuntary movement data thereby to, based on changes in blink parameters as a function of time, perform a determination of subject maintenance of alertness.

One embodiment provides a method including delivering the subject state standardization test whilst simultaneously operating the eyelid monitoring hardware during the defined test period thereby to collect, from the subject, data measurements representative of involuntary eyelid movement parameters during the defined test period.

One embodiment provides a method wherein the steps defined in claim 1 are performed in response to execution of computer readable code via one or more processors of a computer system.

One embodiment provides a method wherein a common hardware unit is configured to deliver the subject state standardization test, collect the subject state standardization test data, provide the eyelid monitoring hardware, and collect the measurements representative of involuntary eyelid movement parameters during the defined test period.

One embodiment provides a hardware system configured to perform a method as described herein, the system including one or more hardware components configured to deliver the subject state standardization test and one or more hardware components configured to collect the measurements representative of involuntary eyelid movement parameters.

One embodiment provides a method for performing a diagnostic test in respect of a human subject, the method including:
delivering a subject state standardization test having controlled test parameters, wherein the subject state standardization test is configured to deliver controlled stimuli to the subject for a defined test period;
collecting response state for the subject state standardization test;
collecting, from the subject, data measurements representative of involuntary eyelid movement parameters during the defined test period;
analyzing the data measurements representative of involuntary eyelid movement parameters during the defined test period thereby to perform a determination in relation to changes in blink event parameters as a function of time.

One embodiment provides a method for performing a maintenance of alertness test in respect of a human subject, the method including:
delivering a subject state standardization test having controlled test parameters, wherein the subject state standardization test is configured to deliver controlled stimuli to the subject for a defined test period;
collecting response state for the subject state standardization test;
collecting, from the subject, data measurements representative of involuntary eyelid movement parameters during the defined test period;
analyzing the data measurements representative of involuntary eyelid movement parameters during the defined test period thereby to perform a determination in relation to the time taken for the subject to progress from an initial state to an objectively defined level of drowsiness.

One embodiment provides a method wherein the step of analyzing the data measurements representative of involuntary eyelid movement parameters includes determining changes in blink event parameters as a function of time.

One embodiment provides a device configured to collect information about a human subject, the hardware device including:
a display screen configured to deliver a visual task;
an input device configured to receive response data from the subject in response to the subject state standardization test;
an image capture module configured to capture image data;
an image processing module that is configured to, via execution of computer executable code, perform analysis of images captured via the image capture module during delivery of the visual task thereby to identify and measure parameters of involuntary eyelid movements by the subject during delivery of the visual task.

One embodiment provides a method for operating a hardware device thereby to collect information about a human subject, the method device including:
operating a hardware device having a display screen thereby to deliver a visual task via the display screen;
monitoring an input device of the hardware device thereby to record response data from the subject in response to the subject state standardization test;
operating an image capture module thereby to capture image data;
operating an image processing module thereby to perform analysis of images captured via the image capture module during delivery of the visual task thereby to identify and measure parameters of involuntary eyelid movements by the subject during delivery of the visual task.

One embodiment provides a wearable hardware unit configured to collect information about a human subject wearing the unit, the unit including:
a body that enables the unit to be worn on the subject's head;
a display device configured to deliver a visual task viewable by the subject;
an input device configured to receive response data from the subject in response to the subject state standardization test; and
an eyelid movement monitoring module configured to identify and measure parameters of involuntary eyelid movements by the subject during delivery of the visual task.

One embodiment provides a unit wherein the eyelid movement monitoring module includes a component configured to perform infrared reflectance oculography.

One embodiment provides a unit wherein the display device includes an augmented reality display.

One embodiment provides a unit wherein the display device includes a retinal projection unit.

One embodiment provides a method for analyzing eyelid movement characteristics in a subject, the method including:
identifying a set of data measurements representative of involuntary movement parameters derived from operation of eyelid motion monitoring hardware during a defined test period;
processing the data measurements thereby to identify a plurality of blink events;
processing the data thereby to, for each blink event, determine blink event parameters; and
processing the data thereby to determine changes in blink event parameters as a function of time.

One embodiment provides a method including categorisation of blink events into a set of predefined classes.

One embodiment provides a method wherein the classes are defined by blink parameter definitions.

One embodiment provides a method wherein the blink parameter definitions include a blink period, such that the blink events are categorised into a predefined set of classes with each class being defined by a respective blink period time range.

One embodiment provides a method wherein processing the data thereby to determine changes in blink event parameters as a function of time includes determining a rate of change in frequency over time of blink events categorised in one or more of the predefined classes.

One embodiment provides a method wherein processing the data thereby to determine changes in blink event parameters as a function of time includes determining a rate of change in frequency over time of blink events categorised in one or more of the predefined classes, and comparing those changes with data benchmarked for one or more sample populations.

One embodiment provides a method including performing a statistical analysis of blink characteristics as a function of time.

One embodiment provides a method including determining rate of change attributes of blink event parameters.

One embodiment provides a method including diagnosing a condition in the subject based attributes of a rate of change of blink event parameters during the defined test period.

One embodiment provides a method for analysis of eyelid parameter data from a human subject, the method including:
monitoring the subject's performance of a defined task that is tailored to place the subject in a set of standardized test conditions for a test period;
identifying a set of involuntary eyelid movement parameters recorded for the subject during the defined test period;
analyzing the subject's performance of a defined task, thereby to identify whether the subject's performance falls within a predefined profile;
in the case that the voluntary subject responses fall within the predefined profile, determining that the involuntary eyelid movement parameters satisfy a diagnostic validity requirement; and
in the case that the voluntary subject responses fall outside of the predefined profile, determining that the involuntary eyelid movement parameters fail to satisfy the diagnostic validity requirement.

One embodiment provides a method for analysis brain function for a human subject, the method including:
monitoring the subject's performance of a defined task that is tailored to place the subject in a set of standardized test conditions for a test period;
identifying a set of involuntary eye and/or eyelid movement parameters recorded for the subject during the defined test period;
analyzing the subject's performance of a defined task, thereby to identify whether the subject's performance falls within a predefined profile;
wherein the standardized conditions are defined thereby to limit a set of factors associated with the subject that influence involuntary eyelid movement parameters, thereby to better isolate involuntary eyelid movement parameters influenced by subject brain function from other influences.

Reference throughout this specification to “one embodiment,” “some embodiments” or “an embodiment” means that a particular feature, structure or characteristic described in connection with the embodiment is included in at least one embodiment of the present disclosure. Thus, appearances of the phrases “in one embodiment,” “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or that comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms “including” or “which includes” or “that includes” as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the present disclosure will now be described, by way of example only, with reference to the accompanying drawings in which:

FIG. 1 illustrates a plot of blink durations for a plurality of test subjects in an “alert” state over the course of a ten-minute observation period, coinciding with a subject state standardization test.

FIG. 2 illustrates a plot of blink durations for a plurality of test subjects in a “drowsy” state over the course of a ten-minute observation period, coinciding with a subject state standardization test.

FIG. 3 illustrates a testing environment where a mobile device having a camera module is used to deliver a subject state standardization test and capture blink data.

FIG. 4 illustrates a graph from which blink events and durations are identifiable.

FIGS. 5A and 5B provide example visualizations of blink data.

### DETAILED DESCRIPTION

Described herein is technology adapted to enable improved collection of involuntary eyelid movement parameters, including collection of eyelid movement parameters to support analysis of neurological factors. For example, this may include methods and systems configured to enable improved analysis of involuntary eyelid movement parameters, including diagnosis of subject neurological conditions and/or other subject attributes from analysis of involuntary eyelid movement parameters. Some embodiments relate to testing, which provide a standardized environment for collection of involuntary eyelid movement data thereby to reduce influence of variable factors, which affect involuntary eyelid movement. For example, the standardized environment influences the subject to adopt a controlled cognitive and/or physiological state, thereby to improve comparability of test results. In some cases, the controlled test parameters include a test parameter, which influences the subject to voluntarily maintain a substantially consistent gaze detection, thereby to minimize eye movement.

Some embodiments described below allow for reliable eyelid movement data collection to be achieved using widely available hardware devices, for example, smartphones and tablet devices, optionally in conjunction with peripheral devices (for example, a handheld input device, such as a button device). Suitable virtual reality headsets may also be used, with these becoming increasingly commonplace. This allows for implementation of extensive data collection programs, which can be of assistance in diagnosis (including individualized diagnosis), research, and other fields. For example, in some embodiments the technology described herein is used in the context of assessing subjects thereby to understand effects of conditions including concussions (or other traumatic injures), degenerative neurological conditions, alertness/drowsiness, and so on. The technology is not limited to any particular purpose of eyelid data analysis.

The embodiments described below refer to analysis of involuntary eyelid movements, also referred to as “blinks” or “blepharon motion,” which may include partial blinks. As used herein, the term “blink” is used to describe an “involuntary blink,” as opposed to a voluntary blink. It is known to differentiate between involuntary blinks and voluntary blinks based on analysis of blink attributes, with voluntary blinks being observably slower.

In overview, one embodiment provides a method for collection of involuntary eyelid movement data from a human subject under controlled conditions, which is achievable using a device such as a smartphone or tablet. In that regard, the method is performed based on execution of software instructions via a hardware device having: (i) a display screen; (ii) a camera module facing in a common direction to the display screen; and (iii) an input device (which may be a touchscreen, or more preferably a handheld peripheral device having in input button).

An app executes on the device thereby to deliver an eyelid movement data collection test. Part of this test is a subject state standardization test, which is delivered via the display screen (or, in some embodiments, delivered audibly). The subject state standardization test is designed to provide a standardized environment for collection of involuntary eyelid movement data, in a manner that reduces influence of variable factors, which affect involuntary eyelid movement. Those factors are widespread, and include cognitive state, physiological state, and environmental conditions. The standardized environment provided in this embodiment influences the subject to adopt a controlled cognitive and/or physiological state by having them engage in a controlled activity, which may be a vigilance-type test, which causes the subject to focus on a simple task that requires focus and attention (this preferably combines with prompting a user to undertake the test in an externally controlled environment). The preferred test influences the subject to voluntarily maintain a substantially consistent gaze detection whilst performing a focused task, thereby to minimize eye movement. In this regard, test results enable improved comparison between data collected in different instances of the test for a common subject and/or between subjects substantially independent of variability responsive to the subjects' respective cognitive and/or physiological states. The expression “substantially independent” acknowledges that there are challenges in completely eliminating all variable influencing factors, but the test environment can reduce effects of a range of such factors as described herein.

The test prompts the subject to provide input, and the app operates to collect, via the input device, voluntary response data for the subject state standardization test. The voluntary response data excludes eyelid movement. The app is additionally configured for collecting, from the subject via the camera module, data measurements representative of involuntary eyelid movement parameters during the defined test period.

The voluntary response data thereby to determine whether the subject's performance of the subject state standardization meets threshold performance requirements, which are defined thereby to allow a determination as to whether the subject adequately engaged in the test (i.e., were they paying sufficient attention to a task they were prompted to perform via the test, based on threshold satisfactory performance). In the case that the voluntary response data meets threshold performance requirements, a determination is made that the data measurements representative of involuntary eyelid movement parameters during the defined test period meet a collection standards requirement. There may a one or more other collection standards requirements, for example:
One embodiment provides a method wherein the hardware device includes or is coupled to one or more sensors that are configured to measure environmental conditions, to verify that those satisfy a further collection standards requirement. The environmental conditions may include one or more of: ambient light; ambient noise; and ambient motion. The ambient motion may include motion of the subject, and/or motion in an area surrounding the subject (this may use both a front and rear facing camera).
One embodiment provides a method wherein the hardware device includes or is coupled to one or more sensors that are configured to measure human physiological conditions, to verify that those meet a further collection standards requirement. The physiological conditions may include one or more of: human movement during the subject state standardization test; human movement prior to the subject state standardization test; and subject heart rate.

In this manner, the technology described herein provides functionality to provide improvements in data collection of eyelid movement parameters, which allows for improved analysis of underlying neurological factors, which influence blinks. Whilst it is known to collect blink data as a secondary measure in other tests, such approaches provide unreliable blink data by creating testing environments that impart a range of unwanted influences on a user's cognitive and/or physiological condition, which influence involuntary blinks and render the resulting blink data of low effectiveness in the context of analyzing underlying neurological conditions and the like.

Additional detail and examples that are in some embodiments incorporated into an app environment based on the preceding example are discussed further below.

### Context to the Disclosed Technology

A human subject's involuntary blinks and eyelid movements known to be influenced by a range of factors, including the subject's behavioral state and brain function. Analysis of data derived from eye and eyelid movements can be analyzed thereby to identify data artefacts, patterns and the like, and these are reflective of the subject's behavioral state, brain function and the like.

In terms of behavioral state, there are many factors that have an effect on involuntary eyelid movements, with examples including: a subject's state of physical activity; a subject's posture; other aspects of a subject's positional state; subject movement; subject activity; how well slept the subject happens to be; levels of intoxication and/or impairment; and others. In terms of brain function, factors that have effects on involuntary eyelid movements include degenerative brain injuries (e.g., Parkinson's disease) and traumatic brain injuries.

The technology described herein is directed to technology that enables collection of eyelid movement data to support analysis of a subject's brain function. This is achieved by an approach that utilizes a data capture environment whereby the subject is placed in a standardized situation, thereby to remove a range of potentially influential factors from involuntary eyelid movements, and enable a deeper focus on particular aspects of brain function that are of interest (for example, brain injuries and/or diseases).

### Enhanced Collection of Eyelid Parameter Data Using State Standardization Test

Some embodiments include methods for data collection and/or analysis of eyelid parameter data from a human subject, which make use of a “subject state standardization test” thereby to improve data collection (for example, in terms of improving comparability of data between subjects by creating an objectively consistent set of underlying neurological factors).

The term “subject state standardization test” refers to a test that is implemented thereby to create a set of standardized conditions under which involuntary eyelid movement data is collected, thereby to enable analysis of bran function from involuntary eyelid movement data independent of a range of factors that would otherwise have substantive influences on those movements. That is, the subject state standardization test is configured and implemented thereby to create standardized conditions under which involuntary eyelid movements are observed and recorded for analysis. This is “state standardization” in the sense of bringing the subject into a standardized condition/state for the purpose of eye/eyelid movement data collection (or at least providing a standardized testing environment, which is configured to influence the adoption of such a state). Some embodiments relate to testing that provide a standardized environment for collection of involuntary eyelid movement data thereby to reduce influence of variable factors, which affect involuntary eyelid movement. For example, the standardized environment influences the subject to adopt a controlled cognitive and/or physiological state, thereby to improve comparability of test results. In some cases, the controlled test parameters include a test parameter that influences the subject to voluntarily maintain a substantially consistent gaze detection, thereby to minimize eye movement.

The state standardization test may be implemented in combination with additional protocols intended to assist in subject standardization. These are in some embodiments prompted via a hardware device that administers the test, and in some embodiments hardware-based sensors re used to monitor compliance with additional protocols (for example, noise sensors, ambient light sensors, physiological sensors such as heartrate sensors, and so on).

In some embodiments, analysis of subject voluntary responses to test stimuli (for example, inputs in response to visual stimuli) are analyzed thereby to validate (or invalidate) reliability of involuntary eyelid movement data. In this sense, the term “reliability” refers to whether, based on the data, there is threshold evidence for a predictive determination that the subject has adequately participated in the subject state standardization test thereby to render the eyelid movement data suitable for the purposes of later analysis (which may, in various embodiments, include either or both of: condition diagnosis; and data set generation for condition diagnosis research/benchmarking). Validation may additionally be based upon analysis of sensor data (for example, noise sensors, ambient light sensors, physiological sensors such as heartrate sensors, and so on).

The state standardization test is a test configured to have controlled test parameters, which delivers to the subject controlled stimuli during a defined test period. The subject provides voluntary responses to those stimuli, and data representative of those responses is recorded in a data system as test voluntary response data. The nature of the test varies between embodiments, and some preferred examples are provided below. By way of example, the responses may be characterized by timestamped event data representing user interactions (for example, pressing a button in response to observation of visual stimuli), which is then correlated against data representing time-specified test response windows).

A key point to note is that the standardization test is performed concurrently with collection if involuntary blink data, but the test is not intended to prompt a user to perform blinks, or cause a user to blink. Rather, the standardization test serves a purpose of standardizing subject conditions during a blink data collection period. This is also clearly distinguished from existing technologies where blink data may be collected during various subject activities that are not specifically intended to standardize subject parameters for the purposes of blink data collection.

Preferred examples of the subject state standardization test include tests corresponding to (or similar/identical to) known forms of vigilance tests or reaction time tests. In fact, some embodiments make use of pre-existing vigilance tests and/or reaction time tests, and/or modified versions of pre-existing vigilance test and/or reaction time tests. However, as discussed further below, key aspects of data collected from the tests for the present purposes vary from the usual data utilized in a vigilance test and/or reaction time test. In general terms, a preferred category of tests leveraged for the present embodiments are traditionally used for the purposes of assessing subject reaction times; in the context of the technology being disclosed herein reaction times are at best a secondary measure, and in some cases not relevant at all. Rather, the primary focus is identification of errors (for example, omission and/or co-mission), thereby to validate that the user has participated in the test (and hence involuntary eyelid data collected during the test can be validated).

In more specific terms, some embodiments utilize a subject state standardization test wherein a visual artefact is displayed at a controlled location on the display screen, and test stimuli is defined by modification of the visual artefact. For example, one test displays a geometric shape at a defined static location on a display screen, and the stimuli is defined by the exchanging of that geometric shape with another geometric shape at the same location. The exchange occurs for a defined period of time, and in certain preferred embodiments that defined period time is consistent across the stimuli. For example, one example along those lines is to display a rectangle on a display screen, and periodically temporarily replace the rectangle with a circle for a set period of time, that providing a stimulus for the test subject to provide an input (of course this is an example only, and a range of other visual artefacts and modifications to visual artefacts may be used). The stimuli are preferably provided on an irregular (for example, pseudorandom) basis.

Preferably, the subject state standardization test is between 5 and 15 minutes in duration, and in some embodiments a test of 10 minutes is used. In some embodiments multiple test variations are made available, for example, a 5-minute test variation and a 10-minute test variations, with the different variations being utilized for respective analytical purposes.

The utilization of a visual artefact at a static location is preferable in cases where eyelid movements are being monitored for analysis, as opposed to eye movements. In these cases, there are advantages associated with removing voluntary eye movements from the test scenario, thereby to further reduce factors that could affect involuntary neuromuscular movements that are associated with blinks. In such embodiments the test is purposely defined thereby to isolate involuntary eyelid movements from voluntary eye movements, and this is readily distinguished from approaches in which a test is delivered for the purpose of recording eye movements such as gaze in response to the test.

In further embodiments, involuntary eye movements are observed, a moving visual artefact is presented, thereby to enable distinguishing of voluntary eye movements (i.e., variations in gaze to follow a moving visual artefact) from involuntary eye movements (such as saccades). Again, this can be distinguished from approaches in which a test is delivered for the purpose of recording voluntary eye movements such as gaze in response to the test; in the context of the technology disclosed herein test is being delivered to identify involuntary eye movements other than those voluntary movements that are elicited as a result of test stimuli

Concurrently with the delivery of the subject state standardization test (i.e., during the test period), eyelid monitoring hardware is operated thereby to observe and record eyelid movements. This enables defining of a set of test involuntary movement data (which is associated with the set of test voluntary subject response data for the subject state standardization test). It is by no means mandatory that the collection of involuntary eyelid data directly correspond to the test period. There should be a substantial overlap, preferably with the eyelid data being collected during the test period. It will be appreciated that in preferred embodiments the collection of involuntary eyelid data substantially corresponds to the test period of the subject state standardization test.

The method includes analyzing the set of test voluntary subject response data, thereby to identify whether the voluntary subject responses fall within a predefined subject state standardization confirmation profile. Based on this analysis:
In the case that the voluntary subject responses fall within a predefined subject state standardization confirmation profile, determining that the associated set of test involuntary movement data meets a diagnostic validity requirement; and
In the case that the voluntary subject responses fall outside the predefined subject state standardization confirmation profile, determining that the associated set of test involuntary movement data fails to meet the diagnostic validity requirement.

As discussed further below, this decision as to whether the set of test involuntary movement data meets or fails to meet the diagnostic validity requirement is utilized as a determiner as to whether that data is used for downstream analysis purposes (which may include, depending on the implementation environment, diagnosis of a neurological condition, other assessment of the subject, identification of a biomarker, or utilization of the data for statistical and/or research purposes).

Analysis based on the state standardization confirmation profile is preferably configured to enable an automated prediction/determination as to whether the subject has for the duration of the test period adopted a standardized state of activity. In some embodiments this includes a prediction that the subject has for the duration of the test period adopted a standardized state of activity is made in the case that the subject satisfies a threshold for successful completion of the test based on a number of errors of omission and/or co-mission.

In this regard, analysis of the set of test voluntary subject response data optionally includes analyzing subject responses to the stimuli, including:
Identifying errors of omission, wherein an error of omission is defined in the case that the subject fails to respond to a given one of the stimuli within a predefined response threshold period;
Identifying errors of co-mission, wherein an error of co-mission is defined in the case that the subject provides a response input that does not correspond to a unique one of the stimuli; and/or
Identifying reaction times based on a time differential between presentation of a given one of the stimuli and a subject response to the stimuli (thereby to validate a relationship between a stimulus and a response, as opposed to perform assessment of reaction time).

In a preferred embodiment analyzing the set of test voluntary subject response data includes, as a primary measure, identifying errors of omission and/or co-mission, and analysis of analysis of response attributes including response time is performed as an optional secondary metric. That is, analysis based on omission and/or commission are useful indicators of whether a user has adopted a desired standardized state during which eyelid measurements can be reliably recorded for downstream purpose; reaction times are more processor intensive to determine and of lesser direct relevance (although in some embodiments they are optionally used, for example, in the context of downstream analysis).

The methods described above provide for valuable enhanced collection of eyelid parameter data, based on the leveraging of a subject state standardization test thereby to remove (or substantially remove) factors that would otherwise influence involuntary eyelid movements. It has been discovered that, by achieving such subject state standardization, there is an ability to obtain standardized data, which can be used to allow more accurate diagnosis of user attributes, such as neurological conditions (for example, alertness/sleepiness, maintenance of alertness, and a range of other conditions). As a result of the standardization of data by these methods, the data is provided in a form that is functional for diagnosis/research purposes, for example, in the context of identifying biomarkers in eyelid movement that are representative of neurological and/or other conditions (such biomarkers, once identified, enabling later diagnosis of those neurological and/or other conditions via the same testing environment).

### Enhanced State Standardization Techniques

In some embodiments, one or more of the enhanced state standardization techniques described in this section are combined with a subject state standardization test as described above thereby to enable additional state standardization.

In some embodiments, delivering the subject state standardization test includes delivering to the subject a set of instructions defining test conditions. For example, where the test is delivered via rending of graphical information on a display screen (or other display apparatus), the set of instructions may be rendered on that screen (optionally in combination with an interactive interface by which a user is caused to confirm that the instructions re being followed). The instructions may include one or more of:
An instruction to engage in a period of prescribed physical activity for a prescribed period prior to commencement of the subject state standardization test. For example, this may include an instruction for the subject to walk around for approximately five minutes.
An instruction regarding ambient lighting conditions during the subject state standardization test. For example, this may be “standard indoor lighting.”
An instruction regarding ambient noise conditions during the subject state standardization test. For example, this may be “a quiet environment.”
An instruction as to body position/posture during the subject state standardization test. For example, this may be “seated still,” and in cases where the test is delivered via a mobile device such as a smartphone “with the smartphone stationary on a table or desk.”

In some embodiments, the subject state standardization test is delivered by a hardware system that includes one or more sensors that are configured to measure test conditions parameters thereby to enable determination of compliance with predefined test conditions requirements. Determination of compliance with the predefined test conditions requirements is then used as additional requirement satisfaction of the diagnostic validity requirement for associated eye and/or eye movement data. That is, a set of involuntary eye and/or eye movement data is determined to satisfy diagnostic validity requirements only where both the voluntary subject responses fall within a predefined subject state standardization confirmation profile, and there is a successful determination of compliance with the predefined test conditions requirements. This optionally includes one or more of the following:
In the case of an instruction to engage in a period of prescribed physical activity for a prescribed period prior to commencement of the subject state standardization test, an inertial measurement unit and/or GPS unit (or other device locating means) may be used to determine (i.e., predict) whether the subject has engaged in the period of prescribed physical activity for the prescribed period. Additionally/alternately, human physiological sensors (for example, heart rate sensors) may be used to collect data during the test preparation and/or during the test itself, thereby to perform a predictive assessment of compliance (actual or practical) with an instruction to engage in a period of prescribed physical activity
In the case of an instruction regarding ambient lighting conditions during the subject state standardization test, a light sensor may be used (for example, a light sensor module used conventionally by a smartphone camera module).
In the case of an instruction regarding ambient noise conditions during the subject state standardization test, a microphone module may be used thereby to monitor for noises above a threshold level.
In the case of instruction as to body position/posture during the subject state standardization test, an inertial measurement unit may be used, optionally in combination with an image capture module and image processing techniques.
In the case of an instruction to undertake the test in an isolated location, in some cases where the hardware unit includes a front and/or rear facing camera, those are optionally used to monitor for movement, thereby to enable assessment of whether the location is indeed isolated. For example, background movements may be identified via image processing techniques (thereby to identify, for example, televisions, passing humans/animals, open windows, and so on, which may all potentially affect eyelid data collection).

It will be appreciated that these are examples only, and other approaches may be used, for example, based on sensor availability on a hardware unit or system used in the context of delivering the state standardization test and/or collecting eye and/or eye movement data (these may be a common hardware unit or separate hardware units, as discussed further below).

### Eyelid Movement Data

This section describes examples of eyelid movement data that are collected and analyzed according to various embodiments. This is provided as general guidance only, and for more detailed disclosure of known techniques for collecting eye and/or eye movement data, reference is made to patent publications including U.S. Pat. Nos. 7,071,831, 7,791,491, 7,815,311 and US 20170119248, each of which are hereby incorporated by cross reference.

In the context of involuntary movement data in the form of eyelid movement data, the eyelid movement data includes a measure of blink attributes for a plurality of blink events during the test period. The measure of blink attributes for a given blink event includes any one or more of: a time period from blink initiation to blink completion (also referred to as a blink duration or blink length); a time period for eye closure motion; a time period during which the eye is closed; and a time period for eye re-opening motion. In some embodiments velocity measurements (which include velocity estimation measurements) are also made, for example, in the context of determining amplitude-to-velocity ratios. This may include a velocity of eye closure motion and/or a velocity of eye re-opening motion.

In a preferred embodiment, the collected blink data includes all or a subset of the following:
An eyelid closing duration (e.g., marked by start time, end time, and amplitude).
An eyelid closed duration (e.g., marked by start time, end time, and amplitude).
An eyelid re-opening duration (e.g., marked by start time, end time, and amplitude).
An amplitude-to-velocity (AVR) ratio opening and/or closing.
An inter-blink interval measured from an end time for a re-opening movement to a start time of a next closing movement.
A blink recurrence interval measured from the start time of a closing movement to the start time of a next closing movement (the inverse of this provides a measure of blink rate).
Attributes derived from statistical analysis of one or more of the above (and/or derivations thereof).

It should be appreciated that data collected depends on a number of embodiment-specific factors, including sensor hardware (as described further below). For example, some measurements such as eyelid motion velocity measurements, amplitude-velocity-ratios, saccades, and the like require high resolution monitoring hardware (for example, hardware configured for infrared reflectance oculography), whereas some embodiments make use of hardware with lesser resolution capabilities (for example, smartphone font-facing camera modules, which often have a frame rate in the order of 60 to 120 Hz, which can be challenging in terms of measuring certain eyelid movement and/or eye movement parameters). Techniques that assist in adopting a sampling method thereby to determine blink artefacts such as AVRs and the like using a camera device are discussed in U.S. patent application Ser. No. 15/318,417.

Some embodiments additionally or alternately include measurement of involuntary eye movement data based on identification and analysis of saccades. As noted above, in some embodiments the subject state standardization test includes delivery of stimuli configured to cause defined voluntary eye movement data thereby to enable identification of involuntary eye movement data associated with saccades.

### Hardware Arrangements

Example hardware arrangements used in various embodiments are described below.

For the purposes of the subject state standardization test, any hardware system including a stimuli delivery component and response component may be used. This may include visual and/or audio type stimuli delivery components. A visual stimuli delivery component is preferred, for example, a display screen that is configured to render a graphical representation of a visual type subject state standardization test (for example, as described further above). This is optionally provided, by way of example, via a television, computer, or mobile device (such as a smartphone or tablet). The response component may include substantially any input device, for example, a button (which is optionally provided on a peripheral device, such as a Bluetooth keyboard or other Bluetooth input device), or in the case of a visual stimuli delivery component in the form of a touchscreen, a region or entire surface of the touchscreen may define the response component (although, as discussed further below, utilization of the touchscreen as an input for stimuli responses is often less preferable given potential to adversely affect state standardization characteristics of the test).

In one example embodiment, the subject state standardization test and eyelid movement parameters are captured by way of a single computing device having a display screen and an image capture unit (e.g., a camera module) facing in a common direction to the display screen (for example, in the case of a smartphone or tablet a front-facing camera, or in the case of a PC or laptop a webcam). In this regard, the image capture unit is provided via hardware device that includes a display screen, and the subject state standardization test is rendered on that display screen. The image capture unit is coupled to a processing unit, the processing unit being configured to execute software instructions that cause processing of image data obtained via the image capture unit, wherein the processing of image data includes image-based identification of one or more eyes of the subject, and for at least one eye, image-based identification of eyelid movement. This is preferably achieved by way of image processing algorithms that are configured to identify eyes and eyelids, and accurately record eyelid position as a function of time (for example, using a mesh/data point tracking arrangement). Image processing algorithms suitable for this purpose are known in the art. In some embodiments a software module is configured to control and/or upscale an image fame rate thereby to optimize measurement resolution. It is advantageous to deliver the subject state standardization test and collect eyelid motion data via a common hardware device on the basis that there is inherent substantial time synchronization between voluntary response data and involuntary eyelid movement data.

One embodiment provides a hardware device configured to collect information about a human subject, the hardware device including: a display screen configured to deliver a visual task; an input device configured to receive response data from the subject in response to the subject state standardization test; an image capture module configured to capture image data; and an image processing module that is configured to, via execution of computer executable code, perform analysis of images captured via the image capture module during delivery of the visual task thereby to identify and measure parameters of involuntary eyelid movements by the subject during delivery of the visual task.

In another embodiment, the eyelid monitoring hardware includes a wearable eyelid parameter monitoring device. A range of devices are known in the art, including devices that make use of electrodes, infrared reflectance oculography (for example, spectacle as described in U.S. Pat. No. 7,815,311, and other forms of devices). In some cases, data from such a device is fed into a computer system, which also receives (directly or indirectly) data resulting from the subject state standardization test.

In a further embodiment, the state standardization test is delivered and eyelid movement data is collected by a common hardware device in the form of a wearable device. One embodiment provides a wearable hardware unit configured to collect information about a human subject wearing the unit, the unit including: a body that enables the unit to be worn on the subject's head; a display device configured to deliver a visual task viewable by the subject; an input device configured to receive response data from the subject in response to the subject state standardization test; and an eyelid movement monitoring module configured to identify and measure parameters of involuntary eyelid movements by the subject during delivery of the visual task.

The eyelid movement monitoring module includes a component configured to perform infrared reflectance oculography, or other eye/eyelid sensor hardware. The display device may include a virtual reality display, an augmented reality display, or a retinal projection unit.

### Analysis Techniques

Some embodiments include, responsive to a determination that a set of test involuntary movement data meets the diagnostic validity requirement, performing analysis of the test involuntary movement data. A range of analysis techniques for both involuntary eyelid movement data and involuntary eye movement data are known in the art, and these are optionally used.

Additionally, an analysis technique may be used, which includes categorization of blink events into a set of predefined classes. The classes are preferably defined by blink parameter definitions. The nature and complexity of classes varies between embodiments, depending on blink parameters that are measured (for example, categories based on one or more of blink duration, opening duration, closed duration, closing duration, opening velocity, closing velocity, amplitude-to-velocity ratio, and others).

Some embodiments, for example, embodiments that make use of a smartphone front-facing camera model for blink measurement, apply a relatively simple but effective blink categorization protocol, which categorizes blink events a predefined set of classes, with each class being defined by a respective blink period time range. An example class schema is as follows:
Category 1 (normal blinks): 90-500 ms
Category 2 (slow blinks): 500-1100 ms
Category 3a (long eyelid closures): 1100-2000 ms
Category 3b (long eyelid closures): 2000-3000 ms
Category 3c (long eyelid closures): 3000+ ms

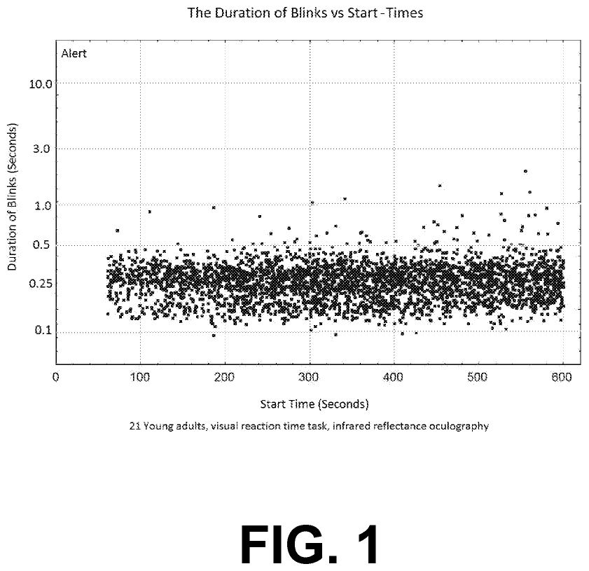

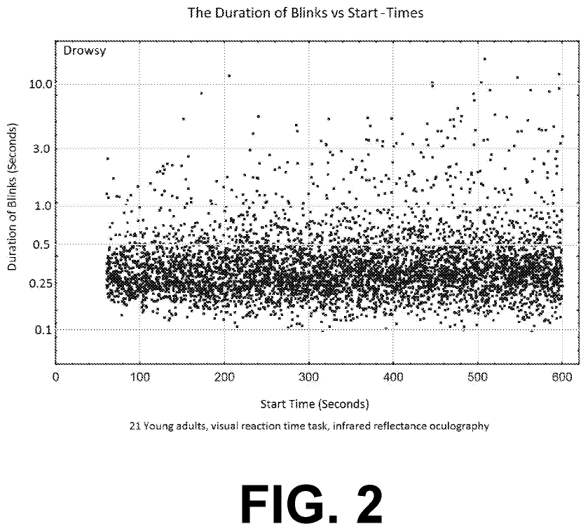

The analysis in some embodiments includes performing a statistical analysis of blink characteristics as a function of time. Examples are shown in FIG. 1 and FIG. 2, which provide a distribution of blink characteristics for a set of test subjects in a “normal” state (FIG. 1) and a “drowsy” state (FIG. 2).

In further embodiments, a class schema is used, which categorizes blink events into a set of categories based on a weighted average of multiple variables (as opposed to duration only). For example, the weighted average is based on a combination of: total duration; opening motion duration; closing motion duration; opening motion velocity; closing motion velocity; amplitude-to-velocity ration, and/or other factors. The weighted averages are in one example used to categories each blink as being:
1. Typical
2. Slow
3. Long eyelid closures

Other categories may also be used; it will be appreciated that utilization of multi-variable weighted averages enables a wide range of category definition possibilities. Based on the categories, analysis is performed based on frequency of each category of blink over a given test period, and more preferably a rate of change of frequency for blinks of each category over the test period.

### Analysis Techniques Based on Rate of Change for Blink Event Parameters

In conjunction with developing and experimenting on blink analysis and with subject state standardization, a new approach to blink analysis has been developed, which includes determining rate of change attributes of blink event parameters as a function of time. As shown in FIG. 2, which illustrates blink duration characteristics as a function of time over the course of a 10-minute data collection period corresponding to a ten-minute subject state standardization test, the frequency of blink events having longer durations increases with time (note the logarithmic nature of the vertical axis). It has been discovered that the rate of change of frequency in blinks of longer durations provides a biomarker capable of representing maintenance of alertness (or the rate at which drowsiness sets in). From this, substantial value associated with performing analysis has been recognized thereby to measure a rate of change in blink attributes as a function of time over a test period, both as a diagnostic tool and as a research tool.

This value is particularly apparent when compared with conventional blink characteristic analysis methods, which focus on overall blink attributes over the course of a test period. By way of example PRECLOS techniques are known in the art, and widely used. PRECLOS in essence measures a percentage of time for which eyes are closed as opposed to open. More specifically, the PERCLOS algorithm, measures a percentage of time either or both eyelids cover the majority pupil (for example, by at least 80%) for a period of time longer than 500 ms, over the course of a test period (which is usually 4-5 minutes). In this way, PERCLOS measures cumulative frequency.

Whereas those former known techniques have merit in the context of detecting late-stage drowsiness, they are not of substantive use in measurement of brain function, for example, diagnosis and/or research into brain injuries and/or diseases. Furthermore, in the context of brain function analysis related to alertness/drowsiness, a PERCLOS type approach is unable to identify attributes such as the beginning of drowsiness and/or the maintenance of alertness, and furthermore are unable to extract a rate of change of blink characteristics over the course of test period, which as discussed here provides useful biomarker information. For the purposes of analysis, a rate of change attributes of blink event parameters optionally includes any one or more of: a point in time during the test period when a change commenced; a rate of change; and an acceleration or deceleration of rate of change over time. The blink event parameters may include any one or more of: blink time period parameters; blink motion rate parameters; and blink frequency parameters. In some embodiments the analysis includes categorization of blink events into a set of predefined classes, and wherein performing the statistical analysis of blink characteristics as a function of time includes determining changes in frequency of blinks categorized into each of the set of predefined classes as a function of time.

Rate of change analysis in relation to blink characteristics over the course of a test (preferably a test providing for subject state standardization as described herein) is optionally used for the purposes of condition/attribute diagnosis (for example, identification of a condition representative biomarker), and/or research. For example, in the context of researching into biomarkers for diagnostic purposes based on data collection across a broad sample group, in the case that the voluntary subject responses fall within a predefined subject state standardization confirmation profile, a determination is made that the associated set of test involuntary movement data meets a diagnostic validity requirement, and the test involuntary movement data is added to a larger data set, and analysis preformed on the larger data set thereby to perform candidate identification for one or more condition-representative biomarkers in eyelid movement data. In this regard, the larger data set includes further sets of test involuntary movement data also determined to meet the diagnostic validity requirement for other subjects. Such diagnosis/research is optionally supplemented with additional data, for example, data collected in response to the subject state standardization test (this, as foreshadowed above, may provide a use for reaction time data).

In some embodiments rates of change are determined for one or more defined sub-periods of a test, for example, an initial test period, or a period commencing following an initial test period. This is because there are certain factors that affect blink parameters during an initial test period—for example, experimentation has revealed a tendency for across-the-board heightened alertness during an initial text period, which declines over time based on factors such as habituation. It is possible to account for factors such as habituation, for example, by collecting user baseline data thereby to assess standard/expected habituation rates for a given subject (or subject fitting defined demographic or other criteria).

### Example Methods

One embodiment provides a method for performing a diagnostic test in respect of a human subject, the method including: delivering a subject state standardization test having controlled test parameters, wherein the subject state standardization test is configured to deliver controlled stimuli to the subject for a defined test period; collecting response state for the subject state standardization test; collecting, from the subject, data measurements representative of involuntary eyelid movement parameters during the defined test period; and analyzing the data measurements representative of involuntary eyelid movement parameters during the defined test period thereby to perform a determination in relation to changes in blink event parameters as a function of time.

Another embodiment provides a method for performing a maintenance of alertness test in respect of a human subject, the method including: delivering a subject state standardization test having controlled test parameters, wherein the subject state standardization test is configured to deliver controlled stimuli to the subject for a defined test period; collecting response state for the subject state standardization test; collecting, from the subject, data measurements representative of involuntary eyelid movement parameters during the defined test period; analyzing the data measurements representative of involuntary eyelid movement parameters during the defined test period thereby to perform a determination in relation to the time taken for the subject to progress from an initial state to an objectively defined level of drowsiness. Preferably, wherein the step of analyzing the data measurements representative of involuntary eyelid movement parameters includes determining changes in blink event parameters as a function of time.

One embodiment provides a method for operating a hardware device thereby to collect information about a human subject, the method device including: operating a hardware device having a display screen thereby to deliver a visual task via the display screen; monitoring an input device of the hardware device thereby to record response data from the subject in response to the subject state standardization test; operating an image capture module thereby to capture image data; and operating an image processing module thereby to perform analysis of images captured via the image capture module during delivery of the visual task thereby to identify and measure parameters of involuntary eyelid movements by the subject during delivery of the visual task.

One embodiment provides a method for analyzing eyelid movement characteristics in a subject, the method including: identifying a set of data measurements representative of involuntary movement parameters derived from operation of eyelid motion monitoring hardware during a defined test period; processing the data measurements thereby to identify a plurality of blink events; processing the data thereby to, for each blink event, determine blink event parameters; and processing the data thereby to determine changes in blink event parameters as a function of time. Preferably this includes categorization of blink events into a set of predefined classes, the classes being defined by blink parameter definitions (for example, a predefined set of classes with each class being defined by a respective blink period time range). Processing the data thereby to determine changes in blink event parameters as a function of time preferably includes determining a rate of change in frequency over time of blink events categorized in one or more of the predefined classes, and/or processing the data thereby to determine changes in blink event parameters as a function of time includes determining a rate of change in frequency over time of blink events categorized in one or more of the predefined classes, and comparing those changes with data benchmarked for one or more sample populations.

### Example Implementation

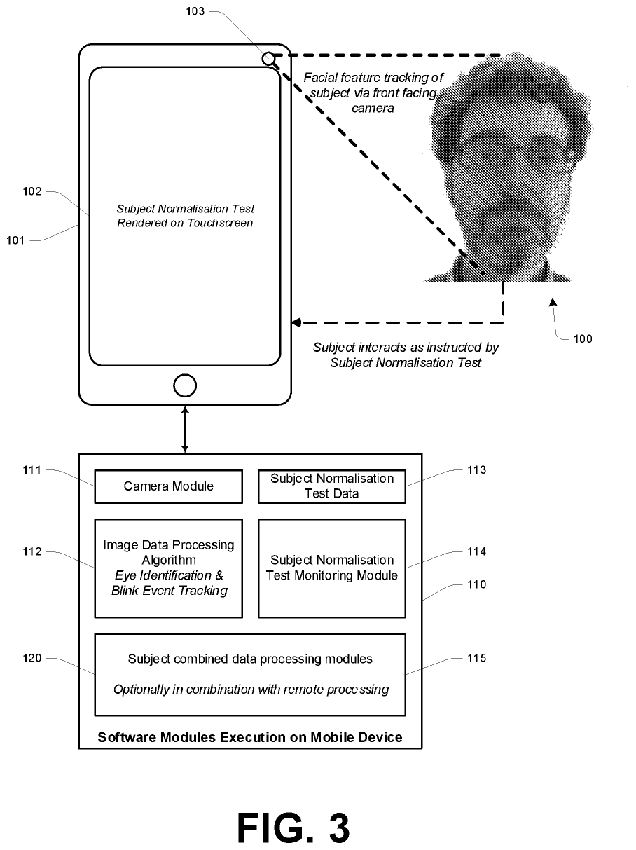

FIG. 3 illustrates an implementation according to one embodiment.

In the example if FIG. 3, a human subject 100 interacts with a computing device 101 (for example, a smartphone or tablet device) having a display screen 102 (for example, a touchscreen) and a camera module 103 (for example, a front facing camera). As of the date of this specification, a preferred hardware device is the iPhone X, and it will be appreciated by those familiar in the art that the iPhone X provides native capabilities relevant to image processing functions described below.

A selection of software modules executing on device 101 are described below. These are illustrated as functionally distinct modules for the sake of convenient illustration.

This system is described by reference to a plurality of modules. The term “module” refers to a software component that is logically separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out.” The statement becomes meaningless if the determination as to whether something is A is not performed.

The term “system” refers to an arrangement where multiple computers, hardware configurations, and devices are interconnected via a communication network (including a one-to-one communication connection). The term “system,” and the term “device,” also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

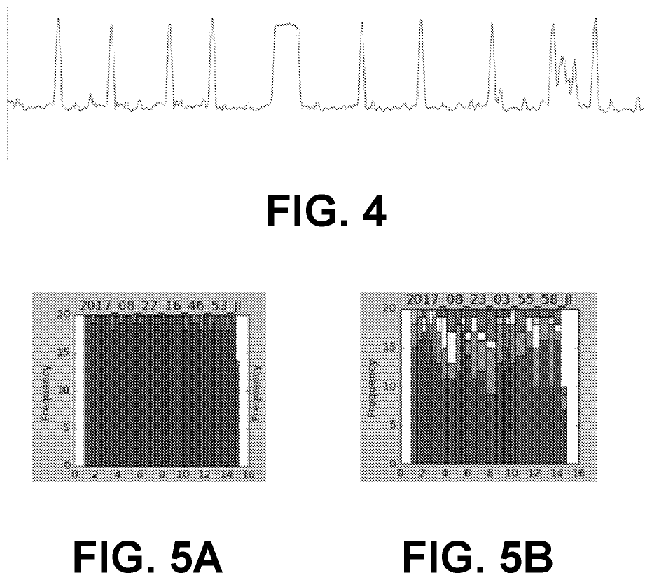

A camera module 111 is responsible for controlling camera module 103, in terms of configuring capture parameters (for example, frame rates and the like, and initiating capture). In this case, it is preferred that capture frame rates are maximized thereby to optimize resolution in tracking the duration of blink events. Data collected via camera module 111 is provided to an image data processing algorithm module 112, which executes one or more image processing algorithms on some or all frames of image data thereby to identify and track the position of facial features. For example, this may include utilization of a tracked data point mesh as shown as an overlay on subject 100 in FIG. 3. Processing module 112 is responsible for identifying blink events via this facial feature tracking, timestamping those events, and recording the duration of each event. This allows the generation of a blink data timeline (for example, as shown in FIG. 4) and blink frequency visualizations (for example, as shown in FIG. 5A and FIG. 5B). These are provided as examples only; it will be appreciated that other visualization approaches may be used, for example, based upon FIG. 1 and FIG. 2, which provide visualization of blink frequency with blink duration data as a function of time over the course of a test period.

In some embodiments, a facial recognition algorithm is used to identify a face, and points on that face that represent: (i) a central position on an upper and/or lower eyelid; and (ii) fixed points relative to the upper eyelid (for example, edges of the eye). The former is used to measure eyelid position, and the latter used to allow scale normalization of data based in changes in relative distance between the user and the camera. That is, a measured difference edges of the eyes may be used to allow normalization of measurements to account to user movements with respect to the camera, such that blink amplitudes are able to be accurately measured.

Camera module 111 and processing module 112 are controlled thereby to operate during a test period during which a subject state standardization test is delivered via display screen 102. In overview, the test is a visual test, which provides controlled stimuli, and a user responds to the stimuli by interacting with an input device (for example, via any of the test approaches described further above). The input device is in some embodiments a connected peripheral having a button, for example, a Bluetooth device (e.g., a Bluetooth keyboard or a simple Bluetooth button device). Alternately, a button provided by the mobile device hardware may be used. In some embodiments the input device is a touchscreen on which the test is displayed. However, it is preferable (but not entirely mandatory) to avoid the use of input devices where user input could result in display screen movement and/or other affects that may adversely affect state standardization effectiveness of the subject state standardization test. Test data is stored by a module 113, and a test monitoring module 114 is configured to record subject responses thereby to enable comparison of those responses with benchmarks (and thereby define a score for the subject state standardization test, which indicates adequate or inadequate performance).

A set of processing modules 120 enable analysis of the blink duration data in combination with state standardization test data. For example, this may provide an alertness score based on analysis of blink duration frequency changes as a function of time, in combination with adequate state standardization test performance. In some embodiments processing functions associated with processing module 120 are performed via remote (e.g., cloud-based) processing facilities.

### Example: Head Injury Assessment Test

One application of technology described herein is in the context of head injury assessment test, which is, for example, utilized in sporting events in the case that a significant head impact has occurred (for instance, in a situation where concussion may be present).

In one embodiment, the test is administered via the system of FIG. 3, although other hardware frameworks described further above may be used (for example, a VR headset). The test includes delivering a subject state standardization test via a display screen (during which whilst blinks are recorded), for a period of between 5 and 10 minutes, which is optionally repeated at defined intervals (which may include usage of shorter intervals initially, for example, to assess whether a player can return to the field, and longer intervals later thereby to assess recovery).

The test preferably utilizes individualized baseline data, which is preferably collected during breaks in physical activities (preferably, where the test is for a particular sport, the subject is tested in breaks during play of the sport to obtain a baseline when no head injury is suspected).

The test has utility in assessing severity of a brain injury, including effects in immediate impairment, which could affect a subject's ability to return to the field in a sporting event.

### Example: Maintenance of Alertness Test

One application of technology described herein is in the context of a maintenance of alertness test, which provides a clinical test to quantify onset time of drowsiness. This is proposed as a replacement for an existing clinical test known as the Maintenance of Wakefulness Test (MWT). This is useful for purposes including: pharmaceutical trials, diagnosis of sleep disorders, evaluation of suitability for tasks, and the like. Such a test is much less cumbersome, time-consuming and expensive than a conventional MWT.

Using technology described herein, a Maintenance of Alertness test is achievable, requiring less time commitment from the subject, greater repeatability and objectivity in results, lower costs, and an ability of administer more flexibly (e.g., at home, at different times, and so on).

In one embodiment, the test is administered via the system of FIG. 3, although other hardware frameworks described further above may be used. The test spans 15 minutes: 5 minutes of alertness state standardization (e.g., walking around) followed by a 10-minute subject state standardization test via a display screen (during which whilst blinks are recorded).

### Example: Blink Data Based Research

In a further example, a software application is distributed to a plurality of subjects, this ap having functionality to provide notifications (for example, time-scheduled notifications) which prompt users to engage in texting thereby to collect eyelid movement data. Data is able to be compared between test attempts for a given user, or across users, thereby to enable comparisons of data collected under common standardizing conditions. This enables collection of large data sets in a distributed and effective manner, which are able to support a range of research initiatives (for example, where additional non-standardized factors affecting one or more subjects are known).

### Conclusions and Interpretation

It will be appreciated that the above disclosure provides technology that enables improved analysis of involuntary eyelid movement parameters, for example, in the context of diagnosing neurological conditions and/or other attributes of a human subject.

It should be appreciated that in the above description of exemplary embodiments of the present disclosure, various features of the present disclosure are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of the present disclosure.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the present disclosure.

In the description provided herein, numerous specific details are set forth. However, it is understood that embodiments of the present disclosure may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B, which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the present disclosure, and it is intended to claim all such changes and modifications as falling within the scope of the invention as defined by the claims. For example, any formulas given above are merely representative of procedures that may be used. Functionality may be added or deleted from the block diagrams and operations may be interchanged among functional blocks. Steps may be added or deleted to methods described within the scope of the present disclosure.

1. A method for collection of involuntary eyelid movement data from a human subject under controlled conditions, wherein the method is performed based on execution of software instructions via a hardware device having: (i) a display screen; (ii) a camera module facing in a common direction to the display screen; and (iii) an input device; the method including:
delivering, via the display screen, a subject state standardization test having controlled test parameters, wherein the subject state standardisation test provides a standardized environment for collection of involuntary eyelid movement data thereby to reduce influence of variable factors, which affect involuntary eyelid movement, wherein the subject state standardization test is configured to deliver controlled stimuli to the subject for a defined test period;
collecting, via the input device, voluntary response data for the subject state standardization test, wherein the voluntary response data excludes eyelid movement;
collecting, from the subject via the camera module, data measurements representative of involuntary eyelid movement parameters during the defined test period, wherein the data measurements representative of involuntary eyelid movement parameters include data describing eyelid position as a function of time for individual blink events;
processing the voluntary response data thereby to determine whether the subject's performance of the subject state standardization meets threshold performance requirements; and
in a case that the voluntary response data meets threshold performance requirements, making a determination that the data measurements representative of involuntary eyelid movement parameters during the defined test period meet a collection standards requirement.

2. The method of claim 1, wherein the standardized environment influences the subject to adopt a controlled cognitive and/or physiological state, thereby to enable comparison between data measurements representative of involuntary eyelid movement parameters between a first subject and a second subject substantially independent of variability responsive to the subjects' respective cognitive and/or physiological states.

3. The method of claim 1, wherein the controlled test parameters include a test parameter that influences the subject to voluntarily maintain a substantially consistent gaze detection, thereby to minimize eye movement.

4. The method of claim 1, wherein the data measurements representative of involuntary eyelid movement parameters include a measure of eyelid position, thereby to enable determination parameters that define blink attributes including eyelid closure times.

5. The method of claim 4, wherein a facial recognition algorithm is used to enable identification of: (i) a central position on an upper eyelid on a detected face; and (ii) at least two fixed points on the detected face; wherein the two fixed points on the detected face are used to enable scaling of measurements of movement of the central position of the upper eyelid thereby to account to changes in relative distance between a user and the camera.

6. The method of claim 1, further comprising processing the data measurements representative of eyelid movement parameters thereby to determine a rate of change for one or more eyelid movement parameters during at least a subset of the time for which the subject state standardization test is delivered.

7. The method of claim 1, wherein a subset of the time for which the subject state standardization test is delivered excludes an initial time segment of the time for which the subject state standardization test is delivered.

8. The method of claim 1, wherein the hardware device includes or is coupled to one or more sensors that are configured to measure environmental conditions, and the method further includes:
processing input data from one of the sensors to determine whether the environmental conditions meet environmental standardisation requirements; and
in a case that the environmental conditions meet environmental standardisation requirements, making a determination that the data measurements representative of involuntary eyelid movement parameters during the defined test period meet a further collection standards requirement.

9. The method of claim 8, wherein the environmental conditions include one or more of: ambient light; ambient noise; and ambient motion.

10. The method of claim 9, wherein the ambient motion includes motion of the subject, and motion in an area surrounding the subject.

11. The method of claim 1, wherein the hardware device includes or is coupled to one or more sensors that are configured to measure human physiological conditions, and the method further includes:
processing input data from one of the sensors to determine whether the human physiological conditions meet physiological standardisation requirements; and
in a case that the physiological conditions meet physiological standardisation requirements, making a determination that the data measurements representative of involuntary eyelid movement parameters during the defined test period meet a further collection standards requirement.

12. The method of claim 11, wherein the physiological conditions include one or more of: human movement during the subject state standardisation test; human movement prior to the subject state standardisation test; and subject heart rate.

13. The method of claim 1, wherein the hardware device is a smartphone or tablet device.

14. The method of claim 1, wherein the subject state standardization test includes a test wherein a visual artefact is displayed at a controlled location on the display screen, and the subject is prompted to provide an input in response to changes in characteristics of the visual artefact.

15. A method for analysis of eyelid parameter data from a human subject, the method including:
accessing a set of test voluntary subject response data, wherein the set of test voluntary subject response data defines a record of voluntary subject responses to stimuli delivered via a subject state standardization test having controlled test parameters, wherein the subject state standardization test is configured to deliver controlled stimuli to the subject for a defined test period;
identifying a set of test involuntary movement data associated with the set of test voluntary subject response data, wherein the set of test involuntary movement data is defined via operation of eyelid monitoring hardware during the defined test period thereby to collect, from the subject, data measurements representative of involuntary eyelid movement parameters during the defined test period;
analyzing the set of test voluntary subject response data, thereby to identify whether the voluntary subject responses fall within a predefined subject state standardization confirmation profile;
in a case that the voluntary subject responses fall within a predefined subject state standardization confirmation profile, determining that the associated set of test involuntary movement data meets a diagnostic validity requirement; and
in a case that the voluntary subject responses fall outside the predefined subject state standardization confirmation profile, determining that the associated set of test involuntary movement data fails to meet the diagnostic validity requirement.

16. The method of claim 15, wherein the subject state standardization test includes a test wherein a visual artefact is displayed at a controlled location on a display screen, and wherein the stimuli is defined by modification of the visual artefact.

17. The method of claim 15, wherein the subject state standardization test is delivered by a hardware system that includes one or more sensors that are configured to measure test conditions parameters thereby to enable determination of compliance with predefined test conditions requirements.

18. The method of claim 17, wherein the one or more sensors include an inertial measurement unit configured to determine hardware motion parameters.

19. The method of claim 17, wherein the one or more sensors include a light measurement sensor configured to determine ambient light parameters.

20. A method for collection of involuntary eyelid movement data from a human subject under controlled conditions, wherein the method is performed based on execution of software instructions via a hardware device having: (i) an output device; (ii) a camera module facing in a common direction to a display screen; and (iii) an input device; the method including:
delivering, via the output device, a subject state standardization test having controlled test parameters, wherein the subject state standardization test is configured to deliver controlled stimuli to the subject for a defined test period;
collecting, via the input device, voluntary response data for the subject state standardization test, wherein the voluntary response data excludes eyelid movement;
collecting, from the subject via the camera module, data measurements representative of involuntary eyelid movement parameters during the defined test period;
processing the voluntary response data thereby to determine whether the subject's performance of the subject state standardization meets threshold performance requirements; and
in a case that the voluntary response data meets threshold performance requirements, making a determination that the data measurements representative of involuntary eyelid movement parameters during the defined test period meet a collection standards requirement.

21. A method for performing a diagnostic test in respect of a human subject, the method including:
delivering a subject state standardization test having controlled test parameters, wherein the subject state standardization test is configured to deliver controlled stimuli to the subject for a defined test period;
collecting response state for the subject state standardization test;
collecting, from the subject, data measurements representative of involuntary eyelid movement parameters during the defined test period; and
analyzing the data measurements representative of involuntary eyelid movement parameters during the defined test period thereby to perform a determination in relation to changes in blink event parameters as a function of time.

22. A method for analyzing brain function for a human subject, the method including:
monitoring the subject's performance of a defined task that is tailored to place the subject in a set of standardized test conditions for a test period;
identifying a set of involuntary eyelid movement parameters recorded for the subject during a defined test period; and
analyzing the subject's performance of a defined task, thereby to identify whether the subject's performance falls within a predefined profile;
wherein the standardized conditions are defined thereby to limit a set of factors associated with the subject that influence involuntary eyelid movement parameters, thereby to better isolate involuntary eyelid movement parameters influenced by subject brain function from other influences.

23. (canceled)
